(19) United States
(12) Patent Application Publication
(10) Pub. No.: US 2007/0155650 A1
(43) Pub. Date: Jul. 5, 2007

(54) Use of cathepsin z inhibitors for the treatment of rhuematoid arthritis and other autoimmune diseases

(75) Inventors: Alan G. Saltzman, Warrington, PA (US); Zhihua Tang, Jamison, PA (US); Vaseem Palejwala, Scotch Plains, NJ (US); Jean Cavallo, Clinton, NJ (US)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: AVENTIS PHARMACEUTICALS INC., Bridgewater, NJ (US)

(21) Appl. No.: 10/596,815

(22) PCT Filed: Dec. 14, 2004
(86) PCT No.: PCT/US04/41815

§ 371 Date: Jun. 26, 2006

Related U.S. Application Data
(60) Provisional application No. 60/533,330, filed on Dec. 30, 2003.

(51) Int. Cl. A61K 38/55; G01N 33/567

(52) U.S. Cl. 514/2; 435/7.2

(57) ABSTRACT

Methods for identifying compounds for treating autoimmune diseases including rheumatoid arthritis. Compounds for treating autoimmune diseases including rheumatoid arthritis. Methods of treatment for autoimmune diseases including rheumatoid arthritis. These methods and compounds are directed to inhibiting the activity of Cathepsin Z.

### BACKGROUND OF THE INVENTION

1. Field of the Invention

The invention relates to the field of treating inflammatory diseases, and in particular to treating inflammatory diseases by inhibiting the activity of Cathepsin Z.

2. Description of Related Art

Cathepsin Z, also known as Cathepsin X, Cathepsin P, and Cathepsin Y, is a human cysteine proteinase of the papain family. Several cysteine proteinases belonging to the papain family are known in the art, including Cathepsin B, Cathepsin H, Cathepsin L, Cathepsin L2, Cathepsin S, Cathepsin K, Cathepsin C, Cathepsin O, and Cathepsin W. Cysteine proteinases of the papain family are known to play an essential role in protein degradation and turnover. Several cysteine proteinases of the papain family are known to play an essential role in lysosomal proteolysis, apoptosis, and antigen presentation. Additionally, cysteine proteinases of the papain family have been implicated in extracellular activities including bone resorption, prohormone activation, rheumatoid arthritis, Alzheimer's disease, pulmonary emphysema, and cancer.

Cysteine proteinases of the papain family are generally known to be translated as preproenzymes which are processed into proenzymes which are then generally targeted into lysosomes via a mannose-6-phosphate signal attached to them. Some of these proenzymes do not end up in a lysosome, but instead are secreted. Some of these proenzymes contain an N-terminal pro region which inhibits proteolytic activity of the mature region until cleavage. Some cysteine proteinases of the papain family require the N-terminal propeptide for correct folding and for stabilization in changing pH conditions. The mature form of cysteine proteinases of the papain family are either a single polypeptide chain or a heterodimer consisting of a heavy chain and a light chain linked by a disulfide bond.

Cathepsin Z is translated as a preproenzyme containing 303 amino acid residues. Santamaria, I, et al., J. Biol. Chem., 273, 16816-16823 (1998). The N-terminal polypeptide of 20 residues of the Cathepsin Z preproenzyme is consistent with known signal peptides. It has been shown that this putative signal polypeptide is cleaved at an Ala-Gly-Ala site to produce a proenzyme containing 283 amino acid residues. Santamaria, I, et al., J. Biol. Chem., 273, 16816-16823 (1998). It has also been shown that the pro region of Cathepsin Z is 41 amino acid residues in length, and that the mature protein is produced by cleaving the proenzyme mostly between the aspartate at position 61 and the leucine of position 62, although a small amount of the purified mature form is cleaved before the aspartate at position 61. The pro region of Cathepsin Z is unusual in that it is shorter than other known pro regions of cysteine proteinases of the papain family. Nagler, D K, and Menard, R, FEBS Lett., 434, 135-139 (1998). The mature region of Cathepsin Z has a predicted molecular weight of 27,280. Santamaria, I, et al., J. Biol. Chem., 273, 16816-16823 (1998). The mature form of Cathepsin Z migrates on SDS/PAGE as a single band with an apparent molecular mass of 33 kDa. The difference between the predicted molecular weight and the apparent molecular weight suggests that the mature form of Cathepsin Z is glycosylated. The mature form of Cathepsin Z is a single chain polypeptide containing two putative N-glycosylation sites comprising the sequence Asn-Tyr-Thr at positions 184 and 224. Santamaria, I, et al., J. Biol. Chem., 273, 16816-16823 (1998). The human Cathepsin Z gene has been mapped to chromosome 20 in the q13 region. Santamaria, I, et al., J. Biol. Chem., 273, 16816-16823 (1998). No other cysteine proteinase of any family has yet been mapped to this region.

The Cathepsin Z gene has been shown to be transcribed in a wide variety of cell tissues, including leukocytes, colon, small intestine, ovary, testis, prostate, thymus, spleen, pancreas, kidney skeletal muscle, liver, lung, placenta, brain, and heart. This wide range of expression suggests that Cathepsin Z is a housekeeping gene involved in normal intracellular protein degradation, similar to the putative functions of Cathepsin B, Cathepsin L, Cathepsin H, and Cathepsin O, and that Cathepsin Z performs this housekeeping on all cell types. It has also been demonstrated that Cathepsin Z is expressed ubiquitously in several human cancer cell lines.

Cathepsin Z acts an exopeptidase which removes one or two amino acid residues at the carboxyl end of polypeptides. Klemencic, I, et al., Eur. J. Biochem. 267, 5404-5412 (2000). No other cysteine proteinase of the papain family is known to function as both a carboxymonopeptidase and carboxydipeptidase. Currently, there is only one other cysteine proteinase of the papain family known to be a carboxypeptidase, Cathepsin B. Cathepsin Z exhibits little or no endopeptidase activity. The optimum pH for Cathepsin Z carboxypeptidase activity is 5.0. It is characteristic of exopeptidases to be inhibited by stefin A, cystatin C, and chicken cystatin. Cathepsin Z activity has been shown to be inhibited by cystatin C, chicken cystatin, stefin A, stefin B, GFG-Sc, and CA-074. Cathepsin Z has been shown to not be inhibited by L-kininogen. This pattern of inhibition is similar to that of the one other cysteine proteinase of the papain family known to be a carboxypeptidase, Cathepsin B. GFG-Sc and CA-704 are known synthetic inhibitors of Cathepsin B. Klemencic, I, et al., Eur. J. Biochem. 267, 5404-5412 (2000). CA-074 has been shown to block Cathepsin B activity in situations related to rheumatoid arthritis, Alzheimer's disease, and cancer. It has been demonstrated that Cathepsin B performs a role in antigen processing. Riese, R, and Chapman, H, Curr. Op. Immun., 12, 107-113 (2000). It has been hypothesized that Cathepsin Z has a role in antigen processing in dendritic cells. Santamaria, I, et al., J. Biol. Chem., 273, 16816-16823 (1998).

The prior art, including references such as Santamaria, I, et al., J. Biol. Chem., 273, 16816-16823 (1998), Nagler, D K, and Menard, R, FEBS Lett., 434, 135-139 (1998), Nagler, D K, et al., Biochemistry, 38, 12648-12654 (1999), Klemencic, I, et al., Eur. J. Biochem. 267, 5404-5412 (2000), and Riese, R, and Chapman, H, Curr. Op. Immun., 12, 107-113 (2000), does not demonstrate or suggest that Cathepsin Z has a role in rheumatoid arthritis. However, results discussed below suggest that Cathepsin Z does play a role in rheumatoid arthritis. It is therefore desirable to inhibit Cathepsin Z function in order to treat rheumatoid arthritis and other autoimmune diseases.

### BRIEF SUMMARY OF THE INVENTION

In one aspect, the invention relates to a method of identifying a compound capable of modulating the activity of Cathepsin Z, having the steps of measuring a cell's level of Cathepsin Z activity in the absence of a candidate compound, introducing the candidate compound, and measuring the cell's level of Cathepsin Z activity in the presence of the candidate compound. In a further aspect, the cell's level of Cathepsin Z activity is determined by measuring the level of antigen presentation. In a further aspect, the cell is an immune cell. In yet a further aspect, the cell is a dendritic cell precursor, an immature dendritic cell, or a mature dendritic cell.

In another aspect, the invention relates to a compound capable of modulating Cathepsin Z activity identified by measuring a cell's level of Cathepsin Z activity in the absence of a candidate compound, introducing the candidate compound, and measuring the cell's level of Cathepsin Z activity in the presence of the candidate compound.

In another aspect, the invention relates to a pharmaceutical for treating an inflammatory disease comprising a compound capable of modulating the activity of Cathepsin Z and a pharmaceutically acceptable excipient.

In another aspect, the invention relates to a method of treating an autoimmune disease having the step of administering the pharmaceutical described above. In a further aspect, the autoimmune disease is rheumatoid arthritis or multiple sclerosis.

### BRIEF DESCRIPTION OF THE DRAWINGS

FIG. 1 is a histogram depicting expression of Cathepsin Z in a wide variety of tissue types.

FIG. 2 is a histogram depicting expression of Cathepsin Z in a wide variety of immune-related cells.

FIG. 3 is a histogram depicting expression of Cathepsin Z in monocytes and immature dendritic cells.

FIG. 4 is a histogram depicting levels of Cathepsin Z mRNA expression in several synovial joints of varying severity in a CIA mouse model.

FIG. 5 is a scatter plot depicting the respective levels of CD68 and Cathepsin Z in several synovium samples.

FIG. 6 is a scatter plot depicting the respective levels of pyrin-8 and Cathepsin Z in several synovium samples.

FIG. 7 is a set of photographs depicting results of in-situ hybridization experiments for localizing expression of Cathepsin Z in rheumatoid arthritis synovium.

FIG. 8 is a set of photographs depicting results of in-situ hybridization experiments for localizing expression of Cathepsin Z in rheumatoid arthritis synovium.

FIG. 9 is a set of photographs depicting results of in-situ hybridization experiments for localizing expression of Cathepsin Z in rheumatoid arthritis synovium.

FIG. 10 is a set of photographs depicting results of in-situ hybridization experiments for localizing expression of Cathepsin Z in normal synovium.

FIG. 11 is a schematic illustration depicting Cathepsin Z's role in antigen processing.

FIG. 12 is a time-course histogram depicting levels of Cathepsin Z mRNA expression at several time points in an EAE model brain.

### DETAILED DESCRIPTION OF THE INVENTION

As illustrated in several of the examples below, experimental results indicate that Cathepsin Z plays a role in antigen presentation, which in turn plays a role in the pathology of autoimmune diseases such as rheumatoid arthritis. Other examples below illustrate methods of modulating the expression of Cathepsin Z in order to treat pathologies associated with autoimmune diseases such as rheumatoid arthritis.

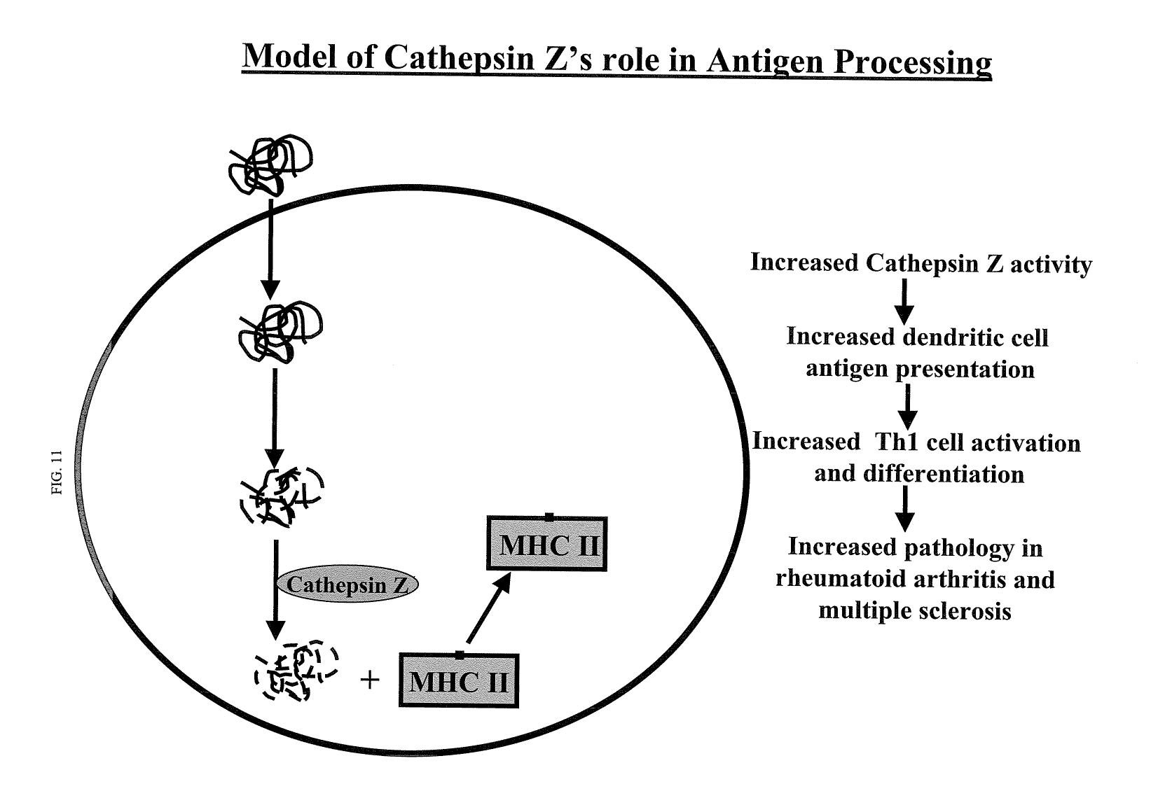

The increased expression of Cathepsin Z in dendritic cells, or increased Cathepsin Z activity in dendritic cells, is associated with increased dendritic cell antigen presentation. The role of Cathepsin Z in antigen presentation is depicted in FIG. 11. An increase in dendritic cell antigen presentation results in increased Th1 cell activation and differentiation. In autoimmune diseases such as rheumatoid arthritis and multiple sclerosis, increased Th1 cell activation and differentiation is correlated with increased pathology. Therefore, interfering with the expression or activity of Cathepsin Z will ameliorate the pathology of an autoimmune disease such as rheumatoid arthritis or multiple sclerosis.

### EXAMPLE 1

### Cathepsin Z Expression Profile

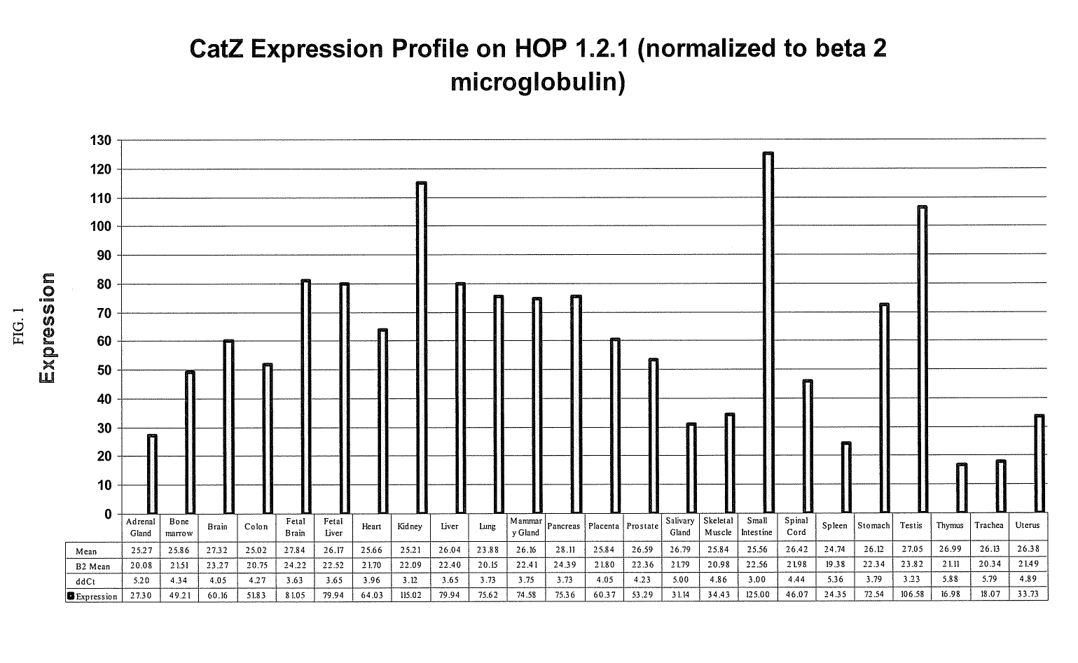

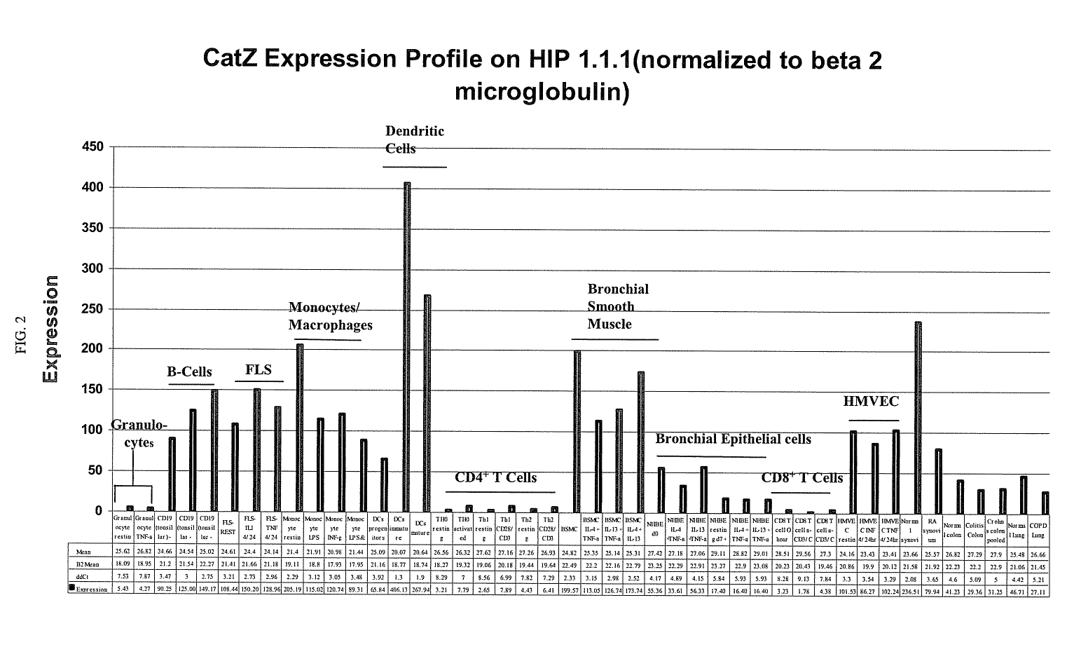

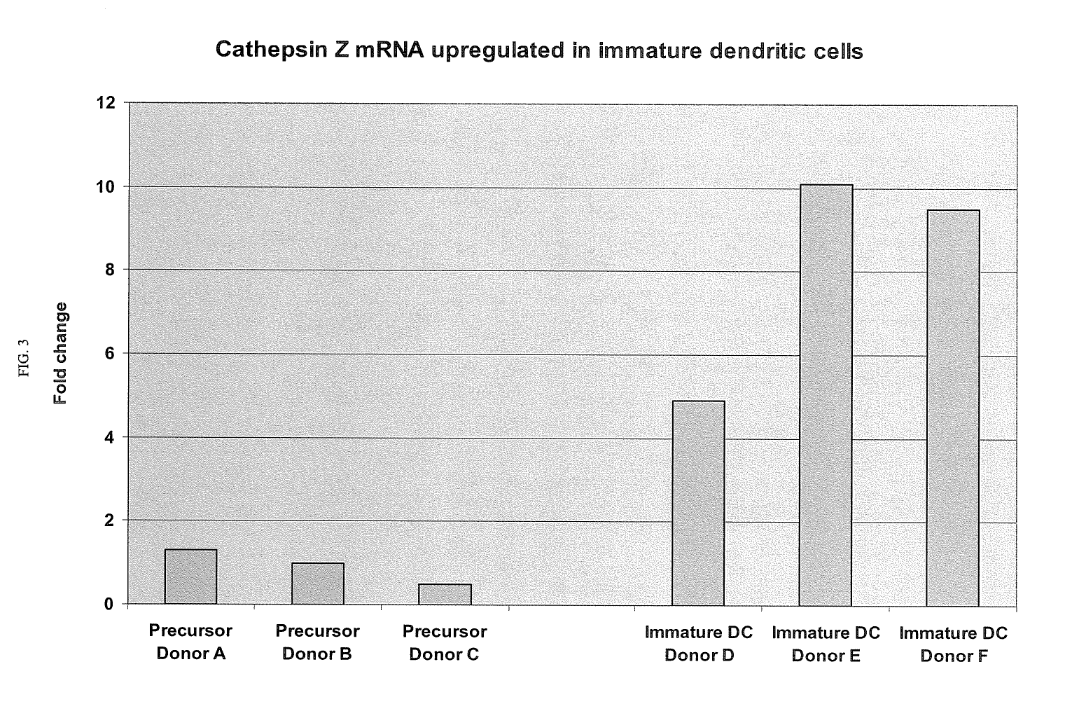

An expression profile of Cathepsin Z was produced by measuring the level of Cathepsin Z mRNA expression in several distinct tissue types. The level of gene expression can be measured quantitatively utilizing a number of established techniques including, but not limited to, Northern blots, RNase protection assays, nucleic acid probe arrays, quantitative PCR including the “TaqMan” assay, dot blot assays and in-situ hybridization. As illustrated in FIG. 1, Cathepsin Z is expressed in a wide variety of tissue types, with especially high expression levels in the kidney, small intestine, and testis. As illustrated in FIG. 2, Cathepsin Z is expressed in a wide variety of immune-related cells, with the exception of CD4⁺ T cells and granulocytes. As further illustrated in FIG. 3, Cathepsin Z expression is upregulated as monocytes differentiate into immature dendritic cells.

### EXAMPLE 2

### Cathepsin Z in CIA Mouse Model

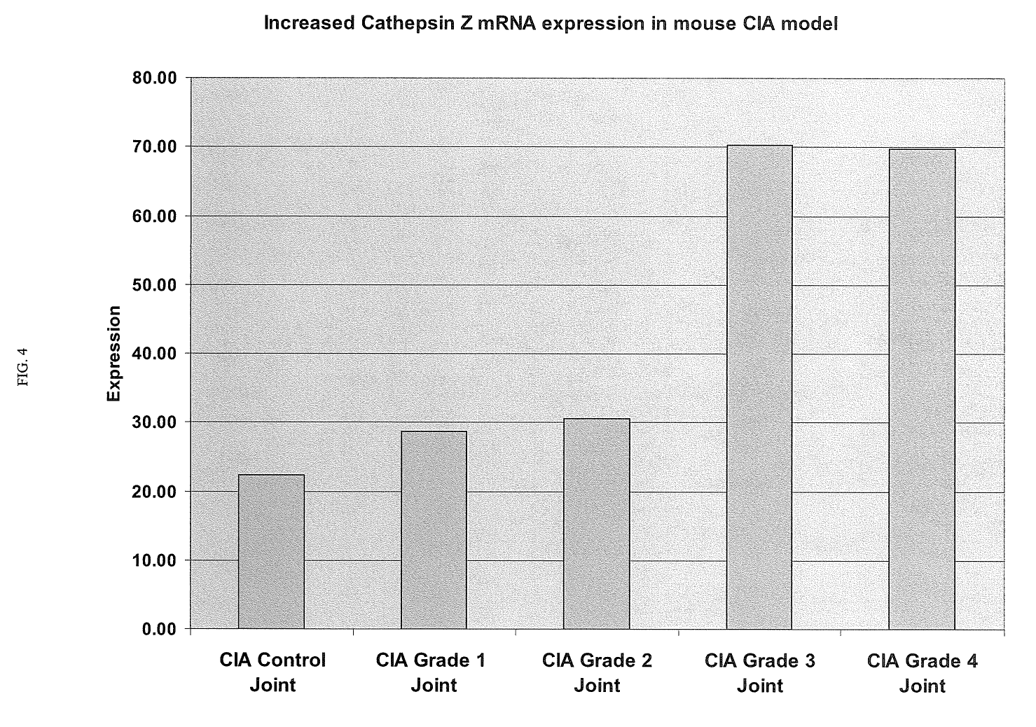

Cathepsin Z exhibits higher levels of expression in several mouse models of disease. The collagen induced arthritis (“CIA”) mouse model is a model of inflammatory synovial disease. In the CIA model, the affected joints are histologically classified according to the following criteria: A grade zero joint is a normal synovial membrane and smooth cartilage surfaces; a grade 1 joint displays synovial membrane hypertrophy and cellular infiltration; a grade 2 joint is exhibits synovial membrane hypertrophy and cellular infiltration plus pannus formation with superficial cartilage erosions; a grade 3 joint exhibits synovial membrane hypertrophy and cellular infiltration and pannus formation with superficial cartilage erosions plus major erosion of the cartilage subchondral bone; and the most severe, a grade 4 joint, exhibits a loss of joint integrity through erosion, massive cellular infiltration, or the presence of ankylosis. FIG. 4 is a graph depicting levels of Cathepsin Z mRNA expression in several synovial joints of varying severity in a CIA mouse model. As illustrated in FIG. 4, the level of Cathepsin Z mRNA transcription increases in a manner roughly proportional to the severity of the synovial joint. The most severe grades of joint are shown to exhibit approximately three times the level of Cathepsin Z expression as a normal joint.

### EXAMPLE 3

### Cathepsin Z in EAE Mouse Model

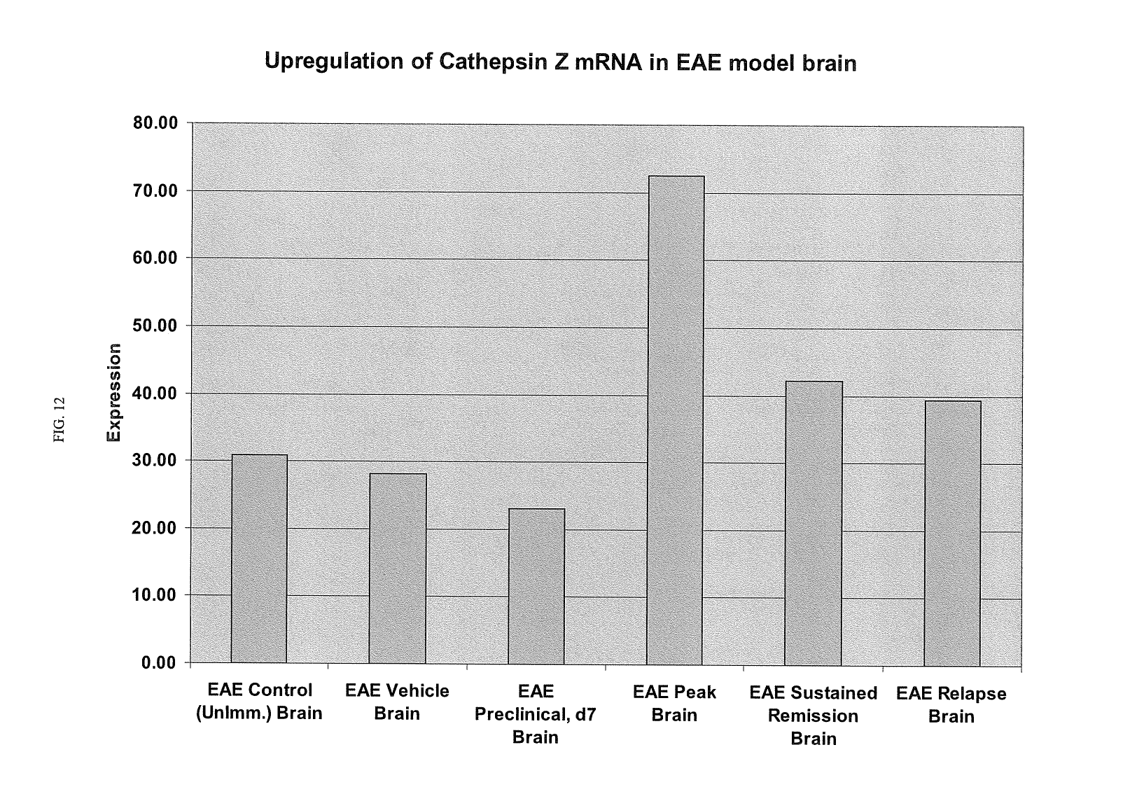

Experimental allergic encephalomyelitis (“EAE”) is a central nervous system autoimmune disease mediated by the action of CD4⁺ T cells, macrophages, and proinflammatory cytokines. FIG. 12 is a time-course histogram depicting levels of Cathepsin Z mRNA expression at several time points in an EAE model brain. As illustrated in FIG. 12, the experimental allergic encephalomyelitis (“EAE”) mouse model exhibits a 3-fold increase in Cathepsin Z mRNA transcription in brain tissue at its peak when compared to normal mice.

### EXAMPLE 4

### Cathepsin Z in Ovalbumin-Challenged Mouse Model

The ovalbumin-challenged mouse model is a model for allergic asthma. An ovalbumin-challenged mouse model has been shown to express Cathepsin Z mRNA at twice the level of untreated mice on Day 4 and Day 7 of the ovalbumin challenge.

### EXAMPLE 5

### Cathepsin Z in Synovia

Synovium libraries have been prepared from rheumatoid arthritis patients and non-rheumatoid arthritis individuals. Specifically, 7 synovium libraries were prepared from rheumatoid arthritis patients and 4 synovium libraries were prepared from non-rheumatoid arthritis individuals. Each synovium library was searched for the presence of clones containing cDNA for Cathepsin Z. Cathepsin Z clones were retrieved from 4 out of the 7 libraries prepared from rheumatoid arthritis patients and none were retrieved from the library prepared from non-rheumatoid arthritis individuals.

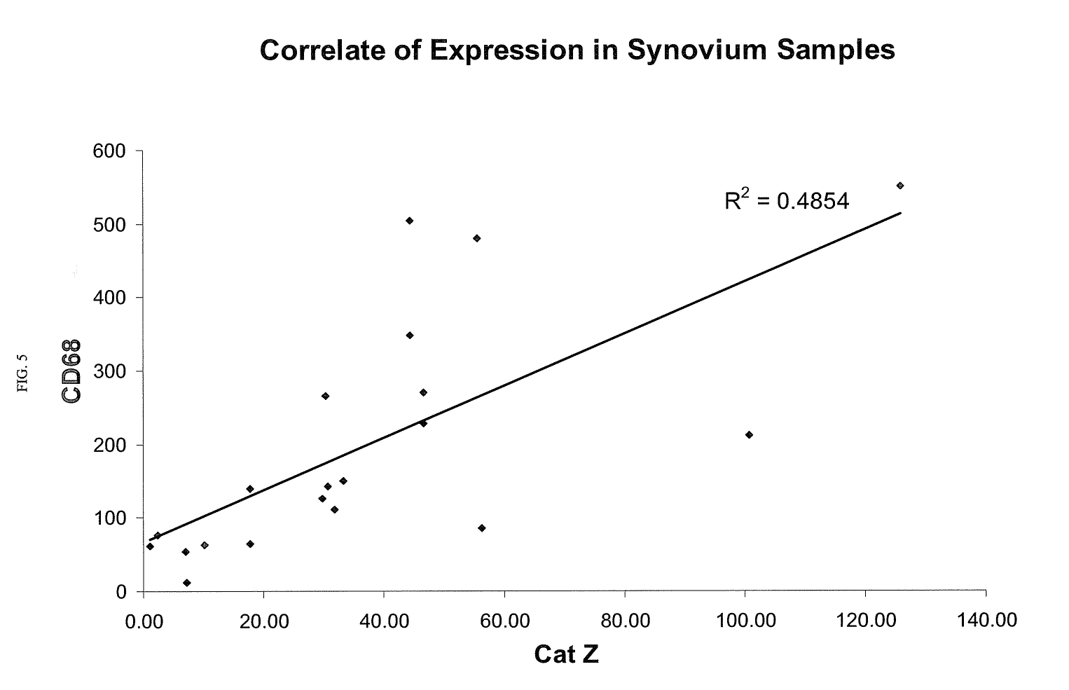

The CD68 marker is found on monocytes and macrophages. The level of CD68 in a synovium is proportional to the number of monocytes and/or macrophages present in that synovium, and hence the severity of arthritis at that synovium. FIG. 5 is a scatter plot depicting the respective levels of CD68 and Cathepsin Z in several synovium samples. As illustrated in FIG. 5, there is a significant correlation between CD68 levels and expression of Cathepsin Z. This result suggests that the level of Cathepsin Z expression in a synovium is roughly proportional to the severity of arthritis at that synovium.

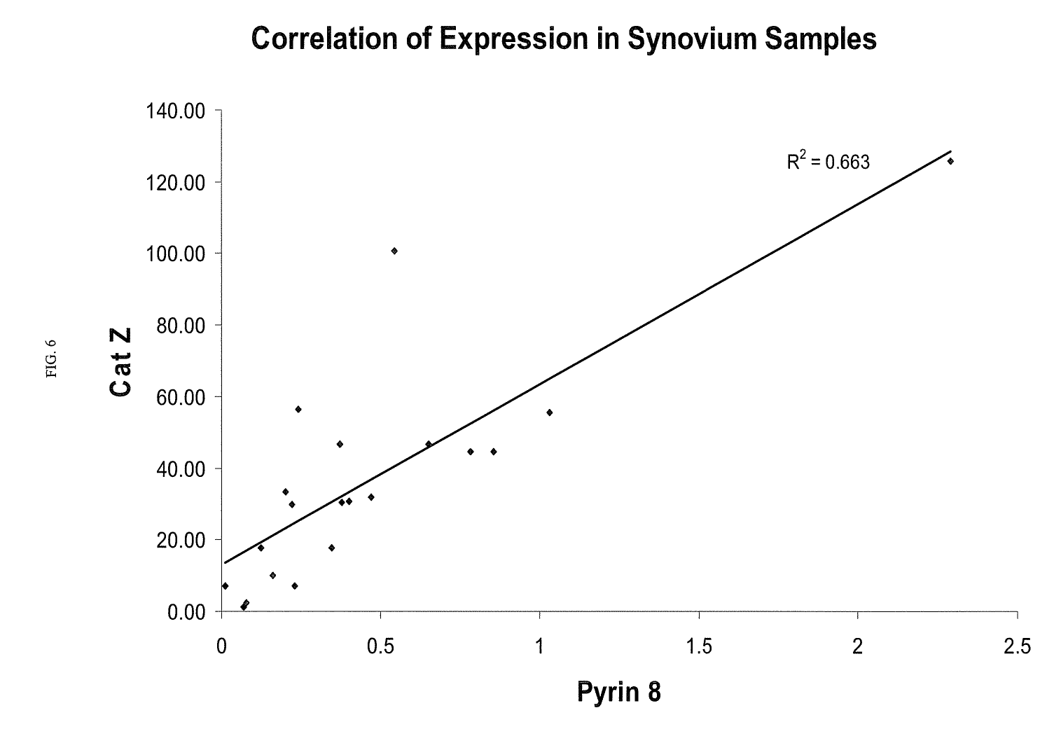

Pyrin-8 is known to be associated with inflammation. The level of pyrin-8 in a synovium is proportional to the level of inflammation. FIG. 6 is a scatter plot depicting the respective levels of pyrin-8 and Cathepsin Z in several synovium samples. As illustrated in FIG. 6, there is a strong correlation between the level of Cathepsin Z expression and Pyrin-8 levels. This result suggests that the level of Cathepsin Z expression in a synovium is roughly proportional to the severity of inflammation at that synovium.

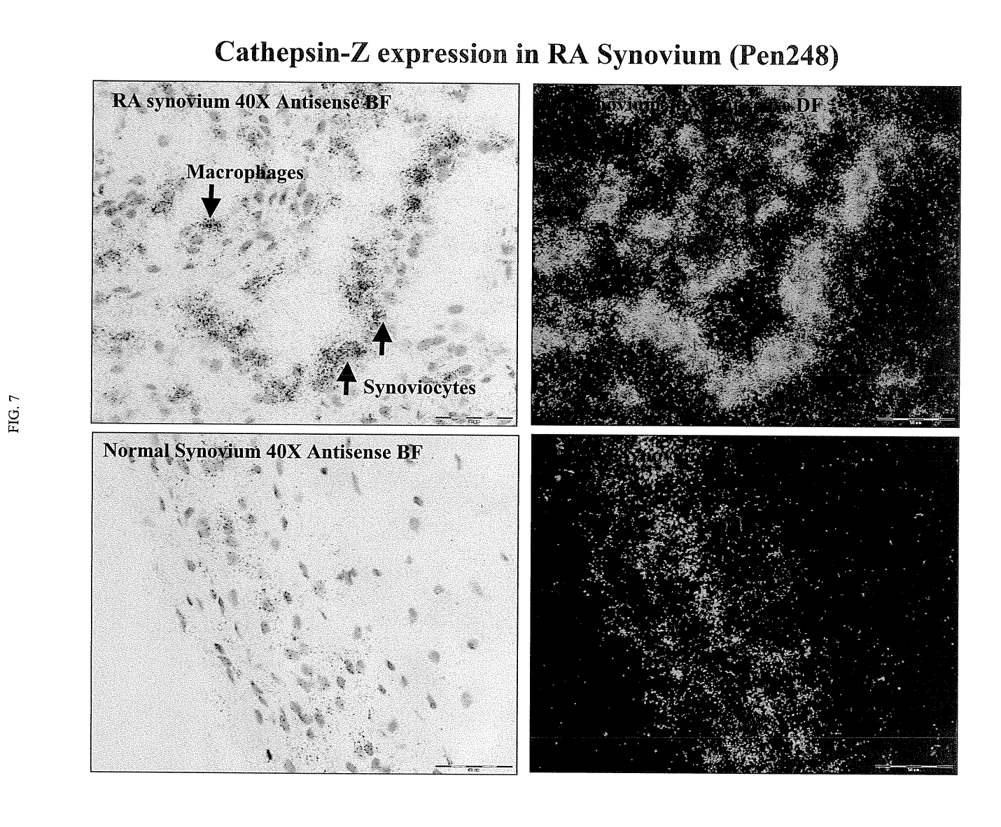

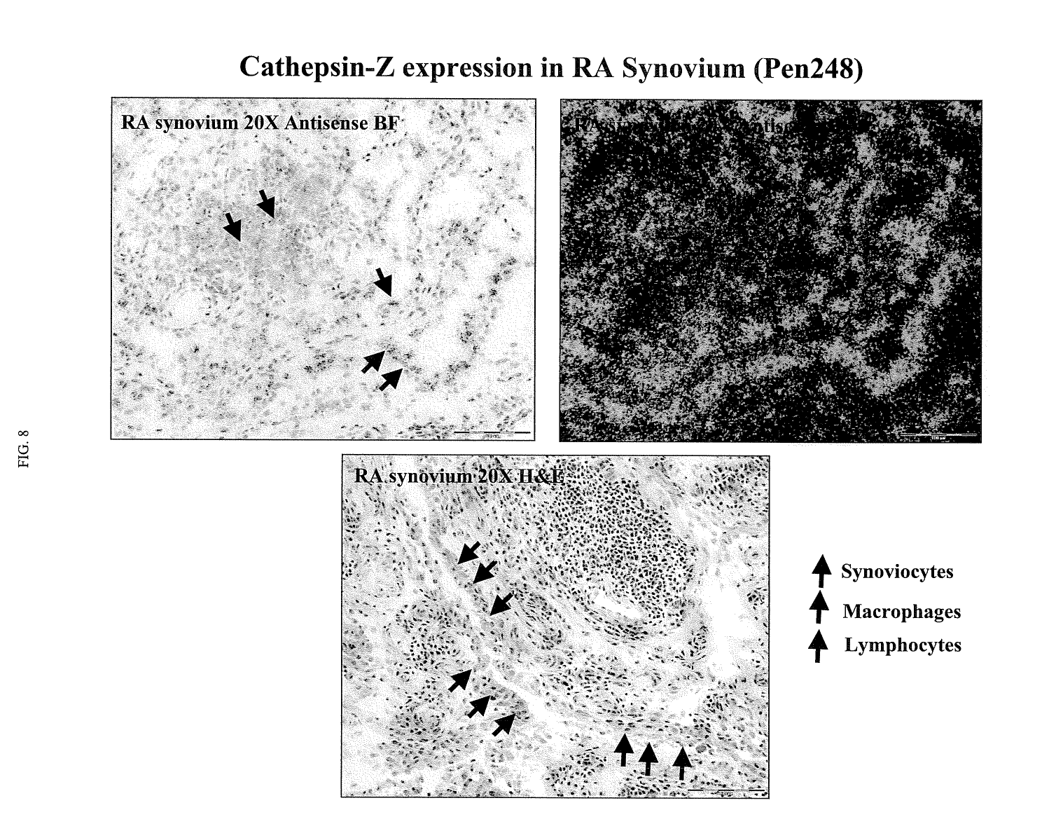

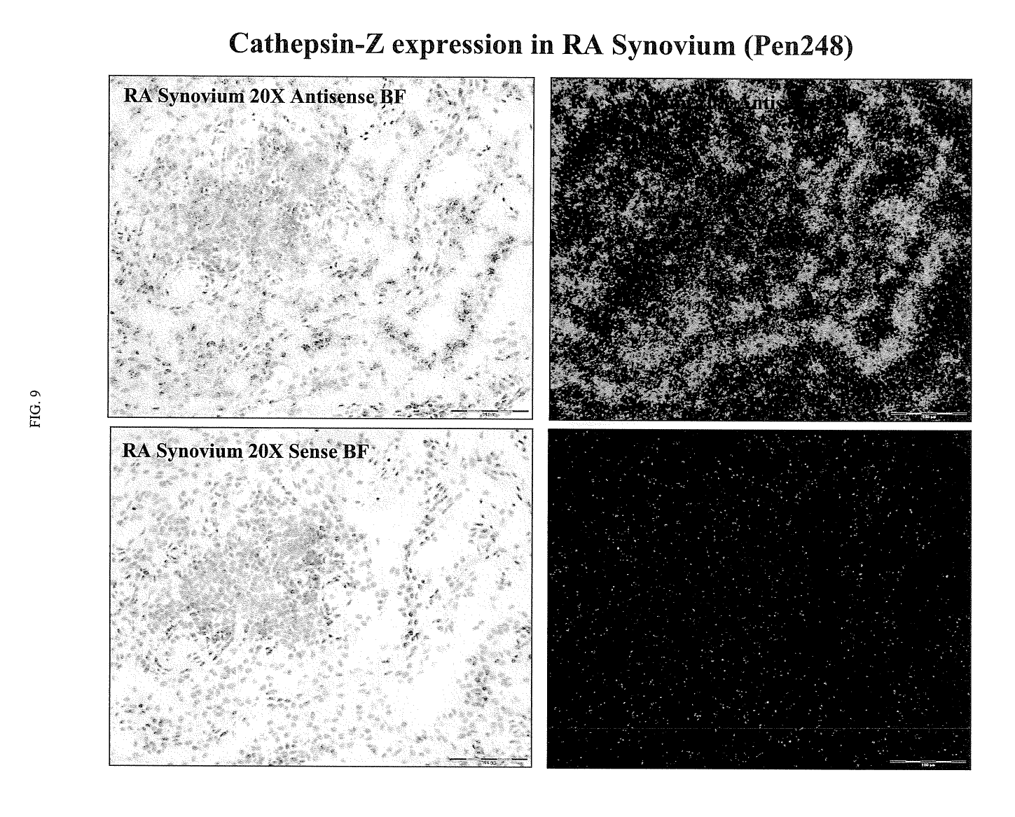

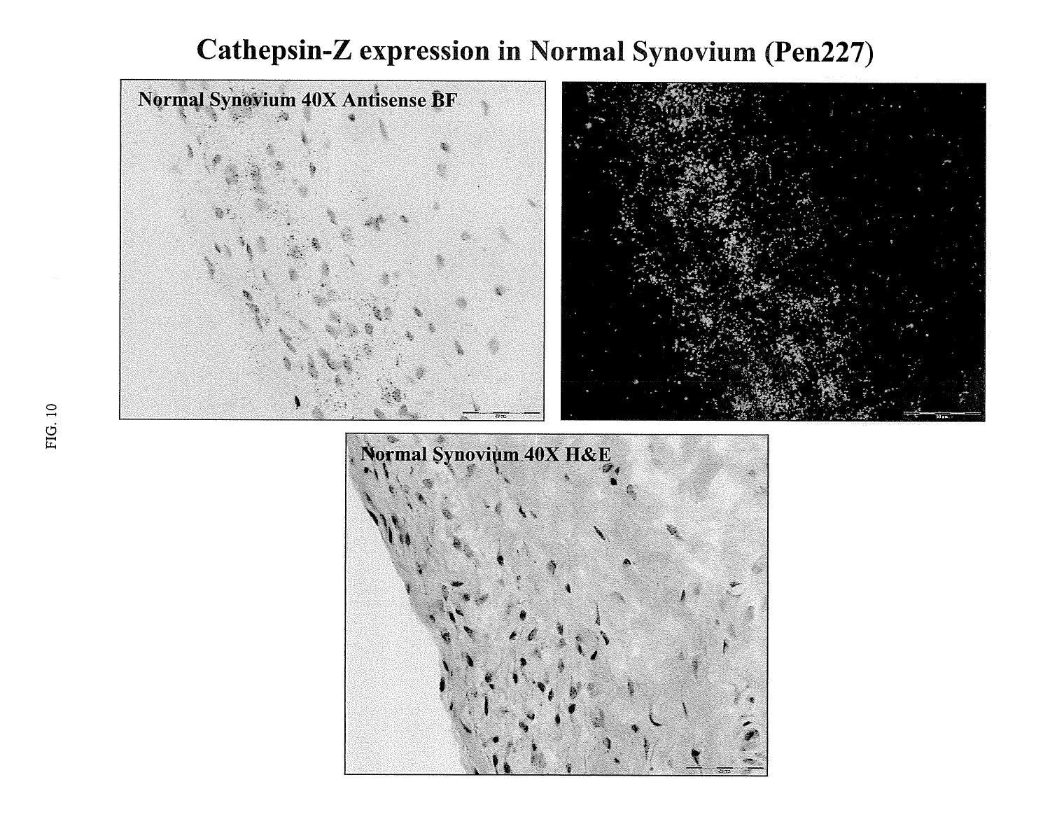

A recent study has shown that a relatively high in-situ hybridization (“ISH”) signal for Cathepsin Z was diffusely or segmentally associated with the hyperplastic synoviocyte population in the specimens of diseased synovial tissue. In contrast, the ISH signal for Cathepsin Z was generally mild with scattered high ISH positive in the relatively quiescent synoviocyte lining of the normal synovial tissue specimen. These discrete cells with high levels of Cathepsin Z expression were present within the infiltrates and were probably macrophages or a small subset of lymphocytes. FIGS. 7-10 depict ISH results for Cathepsin Z, and demonstrate that the Cathepsin Z proteinase is expressed at higher levels in rheumatoid arthritis synovia than normal synovia.

### EXAMPLE 6

### Demonstrating Role of Cathepsin Z in Antigen Processing

Several experiments are performed to demonstrate the role of Cathepsin Z in antigen processing. In these experiments, expression of the Cathepsin Z is reduced or silenced through the use of antisense RNA or RNA interference (“RNAi”).

RNAi refers to the introduction into a cell of double stranded RNA, known as small interfering RNA (“siRNA”), which contains sequences that are antisense to the gene to be silenced or down regulated. Once introduced into the cell, the siRNA is processed into several small single strands of RNA of 21 to 33 nucleotides in length. These small strands of RNA are called guide RNAs. These guide RNAs then associate with a protein complex known as an RNA induced silencing complex (“RISC”). The guide RNA allows the RISC to recognize and bind to the mRNA of the target gene to be silenced. Once the RISC recognizes and binds to the mRNA to be silenced, the RISC then degrades the mRNA.

In one example, antisense RNA or siRNA to Cathepsin Z is introduced into a cell. The cell is then allowed to differentiate. Finally the cell's ability to process antigens is measured. The cell may be an antigen presenting cell, for example. More particularly, the cell may be a human dendritic cell, a human dendritic precursor cell, or a mouse dendritic precursor cell, for example.

In another example, the antisense RNA or siRNA to Cathepsin Z is introduced into the cell via a lentivirus, nucleofection, or a murine stem cell virus, for example.

In yet another example, the cell's ability to process antigens is determined by measuring the cell's capacity to present the common recall antigen tetanus toxin (“TT”). The cell's capacity is determined by measuring autologous T-cell response to tetanus toxin after an incubation period of 5 to 7 days. In another example, the cell's ability to process antigens is determined by measuring the cell's capacity to present quenched FITC-ovalbumin (“DQ-OVA”). In this assay, the cell uptakes the DQ-OVA antigen. When the DQ-OVA antigen is cleaved during antigen processing, the FITC conjugate becomes highly fluorescent. The level of fluorescence, therefore is proportional to the level of antigen processing. Fluorescence can be easily measured by flow cytometry or microscopy.

### EXAMPLE 7

### Treating an Autoimmune Disease by Modulating Cathepsin Z Activity

Several assays are performed to demonstrate the efficacy of treating an organism suffering from an autoimmune disease by modulating Cathepsin Z activity.

In one example, Cathepsin Z activity is modulated by reducing the level of Cathepsin Z mRNA expression. More particularly, Cathepsin Z expression is down regulated through the use of antisense RNA or siRNA to Cathepsin Z, for example.

In yet another example, the organism suffering from an autoimmune disease is a mouse model of disease such as an EAE mouse, an ovalbumin-challenged mouse, or a CIA mouse. In another example, the organism is a human.

In yet another example, the autoimmune disease is rheumatoid arthritis or multiple sclerosis.

### EXAMPLE 8

### Identifying a Compound Capable of Modulating Cathepsin Z

In this example, an assay is performed to determine whether a candidate compound is capable of modulating Cathepsin Z activity in a cell. The cell may be an antigen presenting cell, for example. More particularly, the cell may be a human dendritic cell, a human dendritic precursor cell, or a mouse dendritic precursor cell, for example.

In this example, a baseline level of Cathepsin Z activity is measured in the absence of the candidate compound. Cathepsin Z activity can be measured by measuring the level of the cell's antigen presentation, for example. The candidate compound is then introduced to the cell, and the level of Cathepsin Z activity is measured in the presence of the candidate compound.

The level of Cathepsin Z activity in the presence of the candidate compound is then compared to the baseline level. If these two levels differ, then the candidate compound is identified as capable of modulating Cathepsin Z activity. More particularly, if the level of Cathepsin Z activity in the presence of the candidate compound is lower than the baseline level, the candidate compound is identified as an inhibitor of Cathepsin Z activity.

All references cited herein are incorporated by reference in their entirety.

The invention may be embodied in other specific forms without departing from the spirit or essential characteristics thereof. The foregoing embodiments are therefore to be considered in all respects illustrative, rather than limiting; of the invention described herein. Scope of the invention is thus indicated by the appended Claims, rather than by the foregoing description, and all variants which fall within the meaning and range of equivalency of the claims are therefore intended to be embraced therein.

What is claimed is:

1. A method of identifying a compound capable of modulating the activity of Cathepsin Z in a cell, comprising the steps of:
(a) measuring said cell's base level of Cathepsin Z activity in the absence of a candidate compound;
(b) introducing said candidate compound; and
(c) measuring said cell's level of Cathepsin Z activity in the presence of said candidate compound.

2. The method of claim 1 wherein said cell's level of Cathepsin Z activity is measured by measuring antigen presentation.

3. The method of claim 2, wherein said cell's level of antigen presentation is measured by measuring autologous T-cell response to tetanus toxin.

4. The method of claim 2, wherein said cell's level of antigen presentation is measured by measuring said cell's capacity to present quenched FlTC-ovalbumin.

5. The method of claim 1 wherein said cell is an antigen-presenting cell.

6. The method of claim 5 wherein said antigen-presenting cell is selected from the group consisting of dendritic cell precursor, immature dendritic cell, and mature dendritic cell.

7. A compound capable of modulating the activity of Cathepsin Z identified according to the method of claim 1.

8. A pharmaceutical for treating an inflammatory disease comprising the compound of claim 7 and a pharmaceutically acceptable excipient.

9. A method for treating an autoimmune disease comprising the step of administering the pharmaceutical of claim 8.

10. The method of claim 9 wherein said autoimmune disease is selected from the group consisting of rheumatoid arthritis and multiple sclerosis.
